(19) United States
(12) Patent Application Publication
(10) Pub. No.: US 2019/0232075 A1
(43) Pub. Date: Aug. 1, 2019

(54) ELECTROMAGNETIC DEVICE FOR THERAPIES

(71) Applicant: KENKO PATTO, LLC., Kissimmee, FL (US)

(72) Inventor: GUSTAVO ROCHA KOZUKA, Orlando, FL (US)

(21) Appl. No.: 15/883,669

(22) Filed: Jan. 30, 2018

(51) Int. Cl. A61N 2/02

(52) U.S. Cl. CPC A61N 2/02

(57) ABSTRACT

Physiotherapeutic equipment covered by a plurality of interconnected bipartite enclosures formed by superior covers and inferior covers, being two terminals and three central, in a way that if one terminal is projected to the power cable, has a LED installed indicator of a device that is on and compartment of fusible of safety, in a way that such covers are articulated among them. Internally said device is provided a pair of parallel windings, passing the solenoid in the iron nucleus where five pairs of these are reunited, in an ordinated position in a way that the respective solenoids pass through the alternated current to generate the magnetic field. The structure is protected by resin and a protective overload device is provided as safety, that moves in accordance with the temperature, in a way that such protective overload device is called thermostat and, since it is a device that preserves the temperature in a determined degree, it controls the heat source not to change the temperature towards the propriety that has a tendency to change in accordance with the temperature. The structure is of the associated bimetal type of two metallic plates that have different coefficients of thermodilation, being composed as a carcase, having an internal isolator of ceramics, interconnected through a cable, being filled with epoxy, of the hard rubber type, with an isolator, connection in platinum and terminal.

### BACKGROUND OF THE INVENTION

### Field of the Invention

The present descriptive report is about the invention patent of an innovative electromagnetic device for therapies, the innovative conception is equipped with important technological and functional improvements, in accordance with the most modern concepts of engineering and in accordance with the required standards and specifications, full of particular features and equipped with fundamental requirements of innovation and inventive activity, resulting in a series of real and extraordinary technical, practical and economic advantages.

### Brief Discussion of the Related Art

Currently, there are various analogous products to the object of the present report, but, they are different from this, because they have not objected, in an integral way, all the related and encompassed activities by the demonstrated product.

As mentioned, there are several equipment in the market that have a relation with the electromagnetic device for therapies object of this present patent. However, as it is known by the technicians of the subject, these products do not meet the objectives, for which they were created, being overly archaic, not being functional, unfavorable in its form, easy to erode, in addition to implement its function in an inadequate and/or desired way.

In this sense, some inventors idealized the products object of the patent documents indicated below, but they didn't meet the details above mentioned, especially towards the specificities previously enrolled, and up to this, in short, referenced.

The document of patent PI0607959-8 deposited on Mar. 7, 2006 entitled method for the intensification of pharmacological effects and electromagnetic device for treatment to intensify the pharmacological efficacy that comprehends the application of at least one reactive agent to a structure of target passage, the configuration of at least one wave form that has at least one parameter of wave form, the selection of a value of at least one said parameter of wave form of at least one said wave form to maximize at least one among one relation between signal and noise and one relation between signal of potency and noise, in one structure of target passage, the use of at least one said wave form that maximizes at least one in the said relation between the signal and noise and one relation between potency signal and noise in one structure of target passage where said reactive agent was applied, to generate an electromagnetic signal, and the coupling of said electromagnetic signal to said structure of target passage to modulate said target passage.

The document of the patent P10520604-9 deposited on Sep. 15, 2005 entitled method for the electromagnetic therapeutic treatment to the electromagnetic device of treatment for animals and humans that comprehends the configuration of at least one wave form in accordance with the mathematic model that has at least one parameter of wave form, and at least one said wave form must be coupled to one target passage structure; the choice of a value of at least one said parameter of wave form in a way of at least the said wave form is configured to be detectable in said structure of target passage above the background activity in said structure of target passage; the generation of an electromagnetic signal of at least said configured wave form; the integration of at least one coupling device with a positioning device to be placed in the proximity of said structure of target passage; and the coupling of said electromagnetic signal to said structure of target passage using a coupling device.

The document of the patent P10500441-1 deposited on Feb. 11, 2005 entitled device to treat the body of a patient with the use of an electromagnetic field comprehends a cylindrical coil to generate an electromagnetic field, the cylindrical coil being disposed in a way to accommodate at least part of the body of the patient and submit it to said electromagnetic field, means to activate the coil through a CC pulsed tension, and one support structure comprehending a receipt surface configured to sustain the body, part of the body, inside said cylindrical coil, characterized by the fact of the means being provided to adjust the position of said coil in relation to said receipt surface in at least one direction.

As it will be possible to observe, none of these documents of patent known of the technical status integrally present the constructive characteristics of the electromagnetic device for therapies object of the present patent.

Therefore, through time some studies were made aiming to eliminate these problems and, as a result, this device was developed, aiming the new electromagnetic conception, through where the possibility arises with much more safety to apply therapies in patients, obtaining an integrated equipment with real possibilities of an economic industrialization, minimizing costs, coupling time and labor expenses, in addition to a precise coupling effect, with better results and elevated standard safety.

### SUMMARY OF THE INVENTION

The device object of the present patent is therapeutic magnetized provided of a pair of parallel windings, passing the solenoid in the iron core where five pairs of these are reunited, in an ordered position in a way that the respective solenoids pass through the alternated current to generate the magnetic field.

The structure is protected with resin and an overload protector device is provided for safety, that moves in accordance with the temperature capacity or effect improves the blood circulation and muscle stiffness of the application place.

It improves the human morphology and the position, manipulating the movable part, it is adhered to the rigid place, such as shoulders, hips, legs, etc and is proceeded in the therapy.

### BRIEF DESCRIPTION OF THE DRAWINGS

To complement the present description, in a way to obtain a better comprehension of the characteristics of the present patent, and in accordance with the preferential practical realization of the same, follows the description, in annex, a set of drawings, where, in an exemplified manner that is not restrictive, the following is represented:

FIG. 1 represents the inferior view of the electromagnetic device for therapies object of the present patent;

FIG. 2 represents the lateral view of the electromagnetic device for therapies object of the present patent;

FIG. 3 represents the superior view of the electromagnetic device for therapies object of the present patent;

FIG. 4 represents the view in a profile of the electromagnetic device for therapies object of the present patent;

FIG. 5 represents the cut view of the intern structure of the electromagnetic device for therapies object of the present patent;

FIG. 6 represents the perspective view of the discharge protective device of the electromagnetic device for therapies object of the present patent;

FIGS. 7, 8, 9 and 10 respectively represent the view in elevation, lateral, superior and inferior of the inferior cover of the electromagnetic device for therapies object of the present patent;

FIGS. 11, 12, 13 and 14 respectively represent the elevation, lateral, superior and inferior view of the superior cover of the electromagnetic device for therapies object of the present patent;

FIGS. 15, 16, 17 and 18 respectively represent the elevation, lateral, superior and inferior cut view of the inferior cover of the electromagnetic device for therapies object of the present patent;

FIGS. 19, 20, 21 and 22 respectively represent the elevation, lateral, superior and inferior cut view of the superior cover of the electromagnetic device for therapies object of the present patent;

The FIGS. 23, 24, 25 and 26 respectively represent the elevation, lateral, superior and inferior cut view of the inferior cover of the electromagnetic device for therapies object of the present patent;

FIGS. 27, 28, 29 and 30 respectively represent the other view in elevation, lateral, superior and inferior of the cover of the electromagnetic device for therapies object of the present patent;

FIGS. 31 and 32 represent the silicon plate of the electromagnetic device for therapy object of the present patent;

FIGS. 33, 34 and 35 represent the integrating fuses of the electromagnetic device for therapies object of the present patent; and

FIG. 36 represents the electrical diagram of the electromagnetic device for therapies object of the present patent.

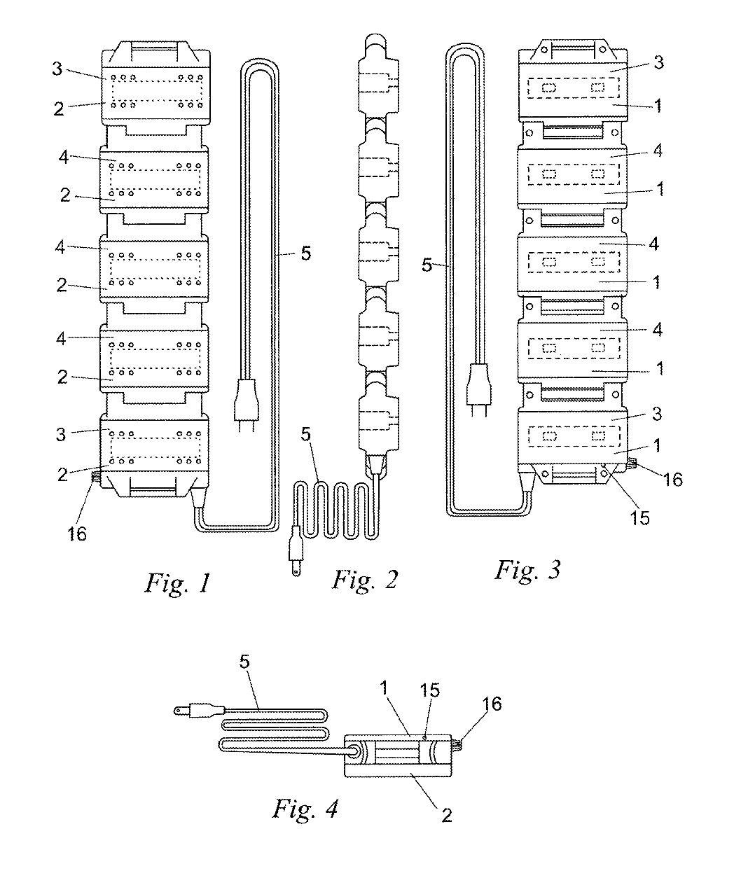

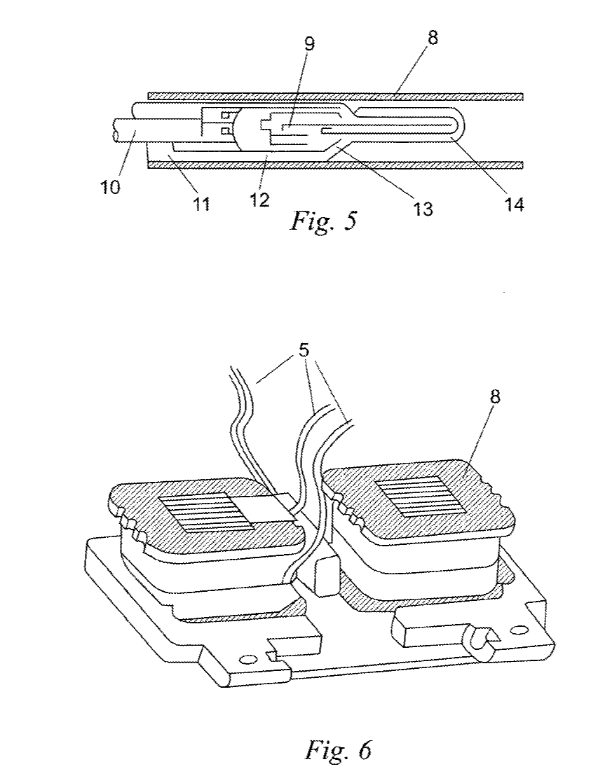

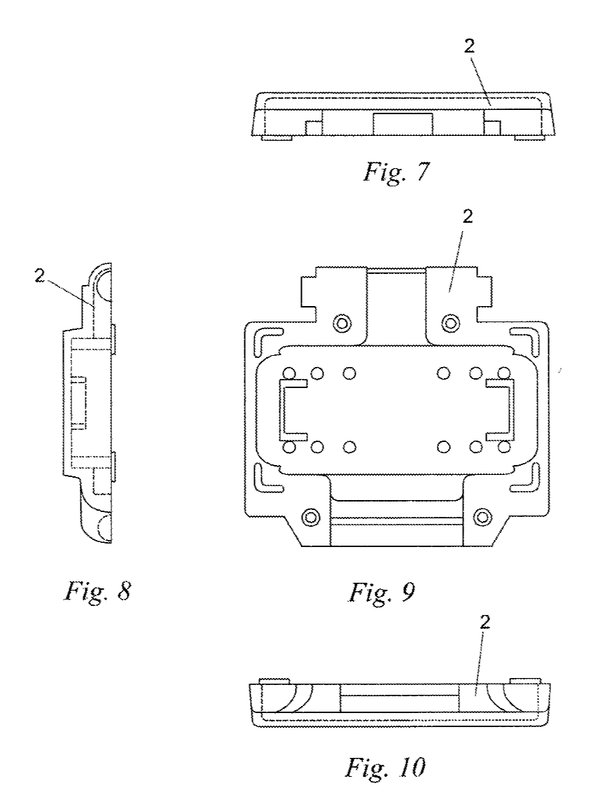

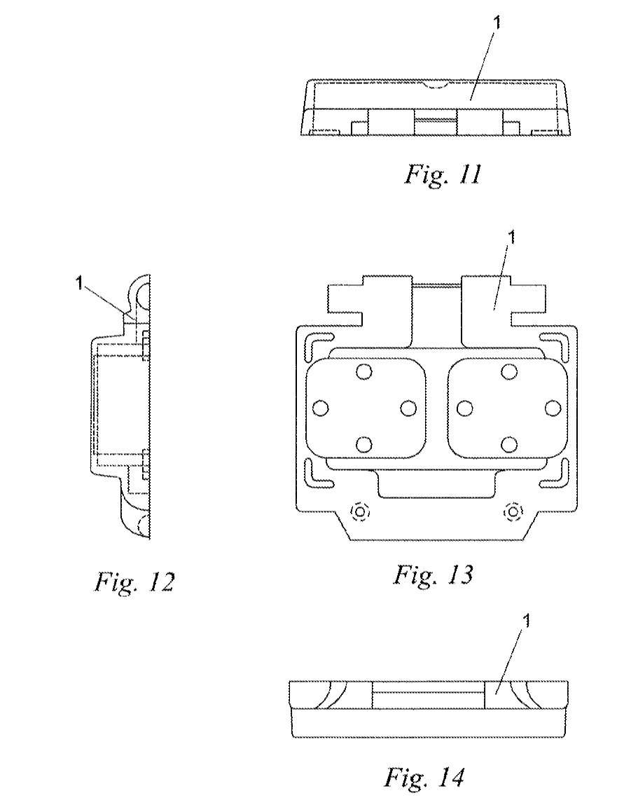

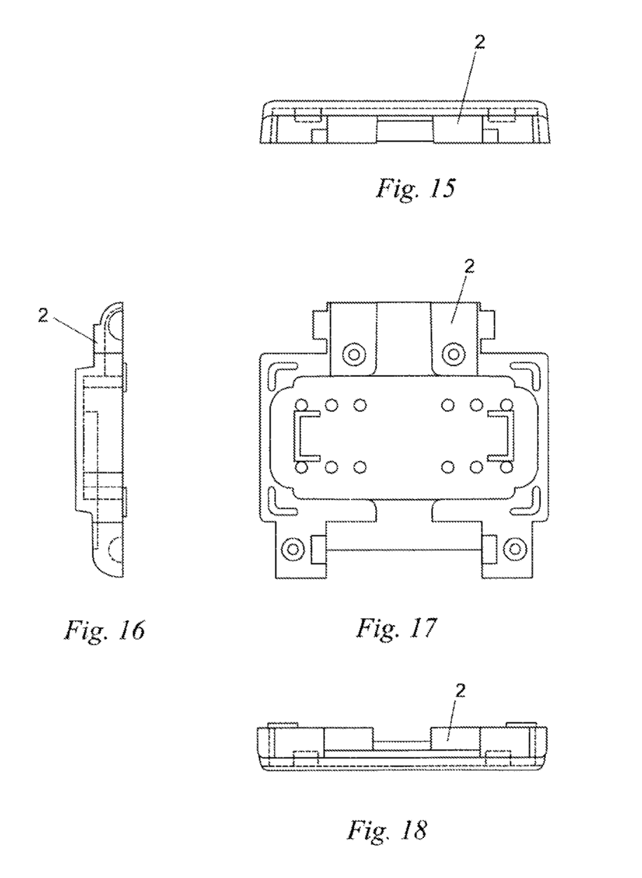

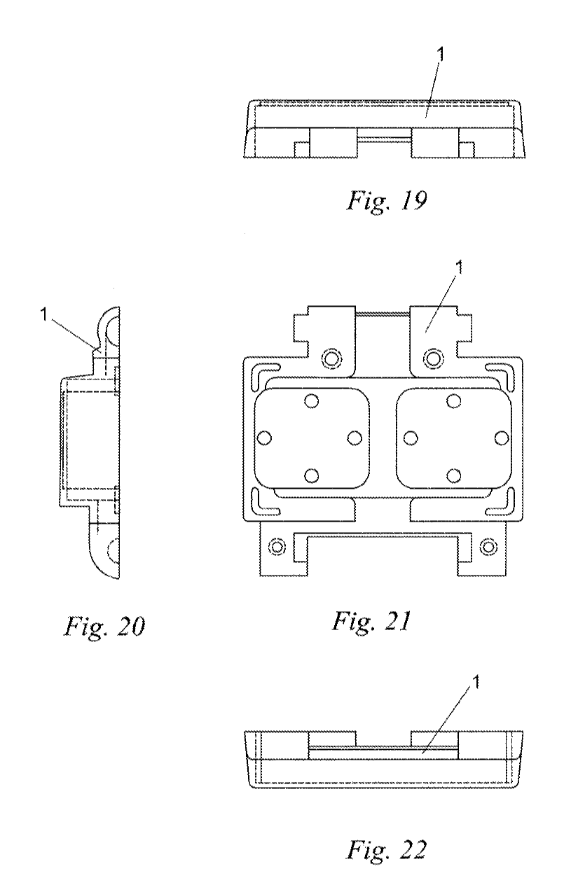

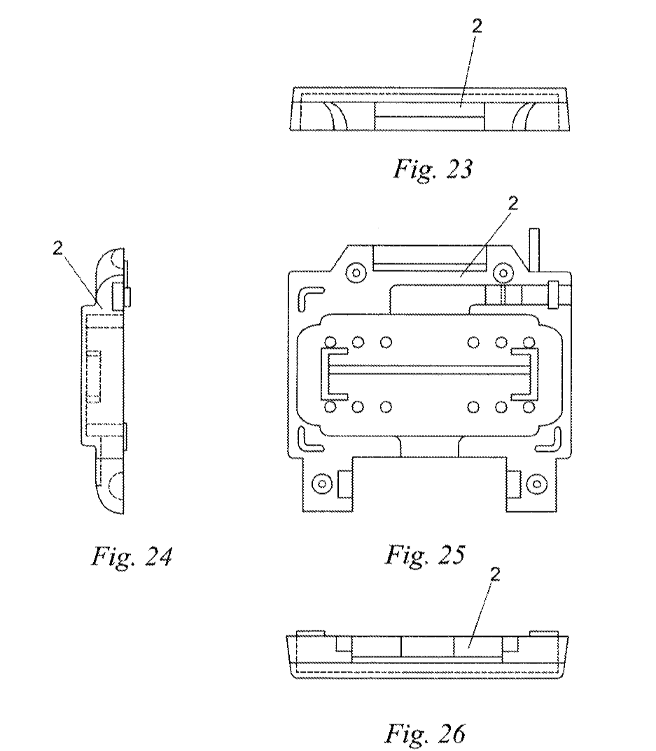

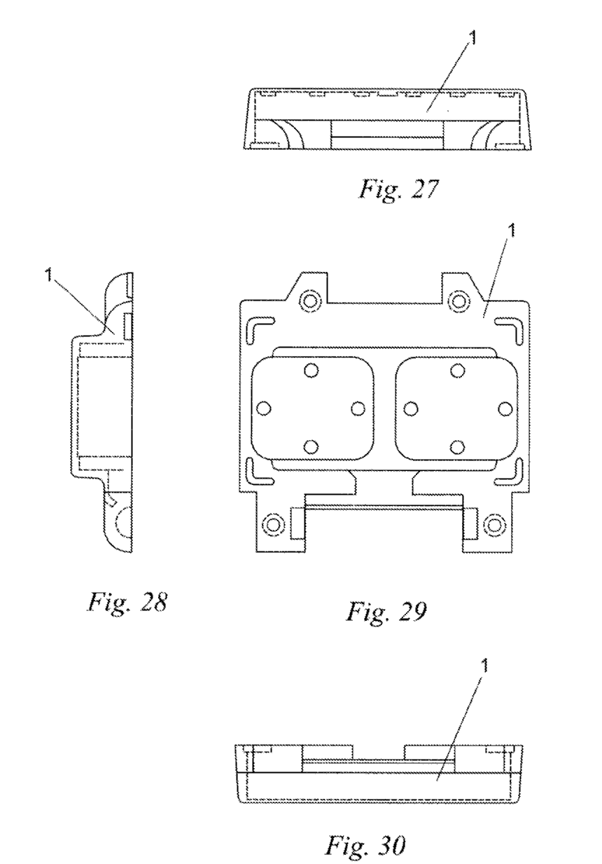

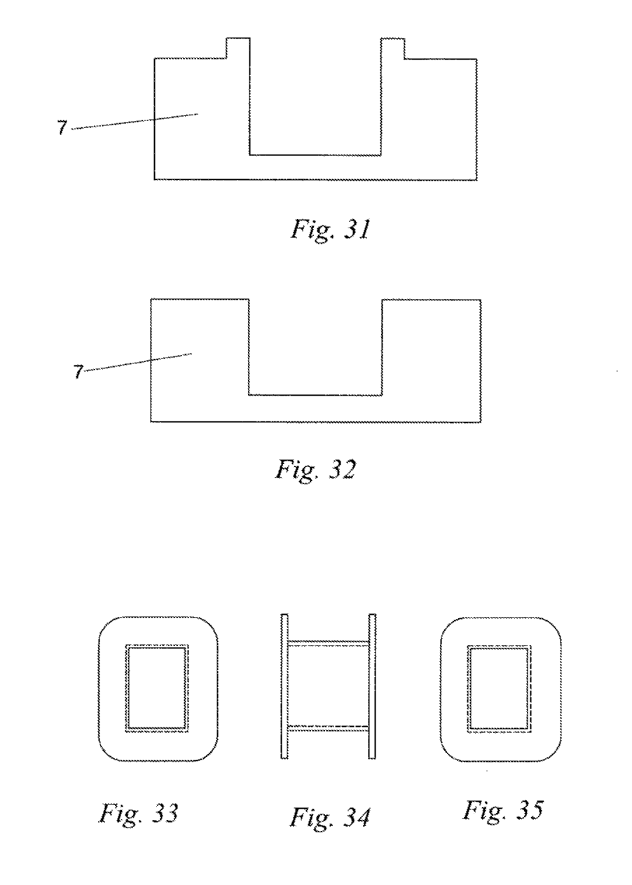

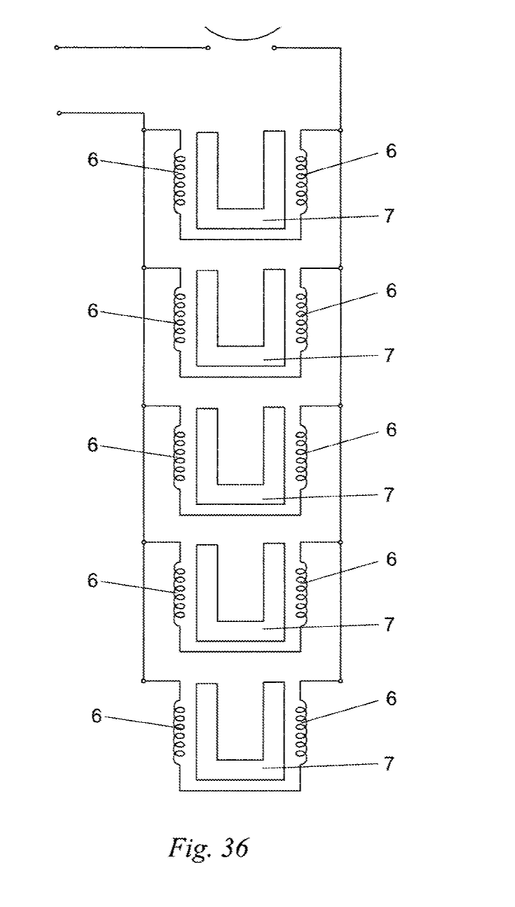

### DETAILED DESCRIPTION OF THE PREFERRED EMBODIMENTS

In accordance with the figures previously mentioned, and in its details, the electromagnetic device for therapies object of the present patent is constituted of a plurality of interconnected bipartite enclosures formed by superior covers 1 and inferior covers 2, being two terminals 3 and three centrals 4, in a way that a terminal is projected to the power cable 5, has a LED 15 indicator of the on device and compartment of fusible 15 of safety, in a way that such covers are articulated among it.

Internally said device is provided of a pair of parallel windings 6, passing the solenoid in the iron nucleus 7 where five pairs of these are reunited, in an ordinated position in a way that the respective solenoids pass through the alternated current to generate the magnetic field.

The structure is protected by resin and an overload protector device is provided, that moves in accordance with the temperature.

The overload protective device is named as thermostat and, since it is a device that preserves the temperature at such degree, it controls the heat source in order to not change the temperature towards the propriety that has a tendency to change in accordance with the temperature.

The type of the structure is an associated bimetal of two metallic boards that have different thermodilation coefficients. It is constituted by a carcase 8, having an internal isolator 9 of ceramic, interconnected by a cable 10, being filled 11 with epoxy, the hard rubber type, with an isolator 12, connection 13 in platinum and terminal 14.

The bimetal has a bracket movement and an installation position of the protective device of overload of the device, in the interval of the symmetrical solenoids, in the internal side, one of them is placed through a tape and thus it can control the saturated temperature.

There is no knowledge of an electromagnetic device for therapies that jointly gathers, all the constructive and functional characteristics previously related, and that directly or indirectly, is or was so effective as the device object of the present patent. Being described and illustrated the present invention it is to be understood that the same can suffer numerous modifications and variations in the realization way, since such modifications and variations are not distant from the invention spirit and scope, such as defined in the reclamation board.

1. An electromagnetic device for therapies, comprising a plurality of interconnected bipartite enclosures formed by superior covers and inferior covers, being two terminals and three central, in a way that if one terminal is projected to the power cable, has a LED installed indicator of a device that is on and compartment of fusible of safety, in a way that such covers are articulated among them.

2. The electromagnetic device for therapies of claim 1, wherein such device is internally provided of a pair of parallel windings, passing the solenoid in the iron nucleus where five pairs of these are reunited, in a position ordinated in a way that through the respective solenoids, an alternated current passes to generate the magnetic field.

3. The electromagnetic device for therapies of claim 1, wherein the structure is protected by a resin and by safety it is provided with an overload protective device that moves in accordance with the temperature, in a way that such workload protective device is named as thermostat and, since it is a device that preserves the temperature of said degree, it controls the heat source in order not to change the temperature against the propriety that has the tendency to change in accordance with the temperature;

4. The electromagnetic device for therapies of claim 1, wherein the structure is an associated bimetal type of two metallic plates that have different coefficients of thermodilation, being composed of a carcase having an internal ceramic isolator, interconnected through a cable, having the filling and epoxy, of the hard rubber type, with isolator, connection in platinum and terminal, in a way that the bimetal has a bracket movement and the installer position of the protective device of overload of the device, in the interval of the symmetrical solenoids, in the internal side, one of them is placed through a tape.
